(19) United States
(12) Patent Application Publication
(10) Pub. No.: US 2007/0027069 A1
(43) Pub. Date: Feb. 1, 2007

(54) Glucan-protein complex extracted from grifola (Maitake)

(75) Inventors: Yuki Masuda, Hyogo (JP); Hazuki Ikumoto, Hyogo (JP); Noriko Kodama, Hyogo (JP); Hiroaki Nanba, Hyogo (JP)

Correspondence Address: DAVIDSON, DAVIDSON & KAPPEL, LLC, 485 SEVENTH AVENUE, 14TH FLOOR, NEW YORK, NY, 10018, US

(73) Assignee: Yukiguni Maitake Co., Ltd., Niigata (JP)

(21) Appl. No.: 11/342,904

(22) Filed: Jan. 30, 2006

(30) Foreign Application Priority Data
Jul. 29, 2005 (JP) 2005-220928

(51) Int. Cl. A61K 38/16; C07K 14/375

(52) U.S. Cl. 514/8; 530/395

(57) ABSTRACT

It is an objective of the present invention to develop a Grifola-derived glucan-protein complex that has a molecular weight lower than that of a conventionally known high-molecular-weight glucan-protein complex and has advanced immunopotentiating activity and antitumor activity. The glucan-protein complex of interest was obtained through the following 1) to 5):
1) a step of thermally extracting mycelia or fruit bodies of Grifola with water; 2) a step of adding alcohol to the obtained water-soluble extract fraction to a final concentration between 20% and 70% by volume, allowing the resulting solution to stand at a temperature between 1° C. and 25° C., and collecting matter floating on or in the solution or adhering to the wall surface of a vessel; 3) a step of dissolving the collected matter in water and collecting non-adsorbed fraction by anion-exchange column chromatography; 4) a step of adding alcohol to the resulting solution to a final concentration between 20% and 50% by volume, allowing the solution to stand at a temperature between 1° C and 25° C., and removing the precipitate that had formed therein; and 5) a step of adding alcohol to the resulting solution to a final concentration between 40% to 99% by volume, allowing the resultant to stand at a temperature between 1° C. to 25° C., and collecting the precipitate that had formed therein.

### BACKGROUND OF THE INVENTION

1. Field of the Invention

The present invention relates to a glucan-protein complex having advanced immunopotentiating activity or antitumor activity that has been extracted and fractionated from mycelia or fruit bodies of “Maitake” mushroom (Grifola). The present invention further relates to an immunopotentiating agent, an antitumor agent, or beverages and foods containing the glucan-protein complex as an active ingredient.

2. Background Art

A polysaccharide having a β-1,6-linked glucose as a main chain and a β-1,3-linked glucose as a branched chain and a polysaccharide having a β-1,3-linked glucose as a main chain and a β-1,6-linked glucose as a branched chain extracted from mycelia or fruit bodies of Grifola have been known to have immunopotentiating activity. (Refer to JP Patent Publication (Kokai) No. 59-210901 A (1984) (Patent Document 1); JP Patent Publication (Kokai) No. 9-238697 A (1997) (Patent Document 2).)

In addition, a method for producing an anticancer substance has been known, wherein “Maitake” (Grifola frondosa), “Choreimaitake” (Dendropolyporus umbellatus), “Tonbimaitake” (Grifola gigantea), or “Masutake” (Laetiporus sulphureus) is extracted with hot water, and the extract is concentrated under reduced pressure so that the resultant is subjected to a combination of a step of precipitation using organic solvents, a step of dialysis for removing low-molecular-weight substances, and a step of removal of impurities by extraction using fat-soluble organic solvents (refer to JP Patent Publication (Kokoku) No. 43-16047 B (1968) (Patent Document 3)).

However, examples of glucan-protein complexes obtained by conventional techniques disclosed in the patent documents above are high-molecular-weight glucan-protein complexes having molecular weights of about 1,000,000. When examining the detailed immunopotentiating activity mechanisms thereof at a cellular level, the fact that such complexes have large molecular weights means that the main active structures thereof cannot be specified. In addition, since such glucan-protein complexes have high molecular weights, they cannot be administered intravenously.

[Patent Document 1] JP Patent Publication (Kokai) No. 59-210901 A (1984)

[Patent Document 2] JP Patent Publication (Kokai) No. 9-238697 A (1997)

[Patent Document 3] JP Patent Publication (Kokoku) No. 43-16047 B (1968)

### SUMMARY OF THE INVENTION

It is an objective of the present invention to develop a Grifola-derived glucan-protein complex that has a molecular weight lower than that of a conventionally known high-molecular-weight glucan-protein complex and has advanced immunopotentiating activity and antitumor activity.

Inventors of the present invention have newly obtained a glucan-protein complex having a molecular weight of 10,000 to 150,000 by fractionation of a hot water extract of Grifola, and have found that the obtained substance contains a β-1,6 linkage and a β-1,3 linkage in the structure thereof. Thus, a low-molecular-weight glucan-protein complex that is a useful immunopotentiating agent or an antitumor agent was obtained. The obtained glucan-protein complex is similar to glucan-protein complexes described in the patent documents above in terms of polysaccharide structure, while on the other hand, is obviously different therefrom in terms of molecular weight and protein content.

The present invention relates to the following (1) to (6):

(1) a glucan-protein complex produced by the following 1) to 5):

1) a step of thermally extracting mycelia or fruit bodies of Grifola with water;

2) a step of adding alcohol to the obtained water-soluble extract fraction to a final concentration between 20% and 70% by volume, allowing the resulting solution to stand at a temperature between 1° C. and 25° C., and collecting matter floating on or in the solution or adhering to the wall surface of a vessel;

3) a step of dissolving the collected matter in water and collecting non-adsorbed fraction by anion-exchange column chromatography;

4) a step of adding alcohol to the resulting solution to a final concentration between 20% and 50% by volume, allowing the solution to stand at a temperature between 1° C. and 25° C., and removing precipitate that had formed therein; and

5) a step of further adding alcohol to the resulting solution to a final concentration between 40% to 99% by volume, allowing the resultant to stand at a temperature between 1° C. to 25° C., and collecting precipitate that had formed therein;

(2) the glucan-protein complex described in (1), the glucan to protein ratio of which is between 70:30 and 99:1;

(3) the glucan-protein complex described in (1) or (2), which is obtained from “Maitake” (Grifola frondosa), “Shiromaitake” (Grifola albicans Imaz.), or “Choreimaitake” (Dendropolyporus umbellatus);

(4) an immunopotentiating agent containing, as an active ingredient, the glucan-protein complex described in (1), (2), or (3);

(5) an antitumor agent containing, as an active ingredient, the glucan-protein complex described in (1), (2), or (3); and

(6) beverages and foods containing the glucan-protein complex described in (1), (2), or (3).

The inventors of the present invention have developed an antitumor substance extracted from Grifola as described in Patent Document 2 above. Considering the main points of the aforementioned invention, the invention relates to a glucan-protein complex produced by the following (1) to (3): (1) a step of thermally extracting mycelia or fruit bodies of Grifola with water; (2) a step of adding alcohol to the obtained water-soluble extract fraction to a final concentration between 20% to 70% by volume, allowing the resulting solution to stand at a temperature between 1° C. to 25° C., and removing matter floating on or in the solution or adhering to the wall surface of a vessel; and (3) a step of adding alcohol to the resulting solution to a final concentration between 80% to 99% by volume, allowing the resultant to stand at a temperature between 1° C. to 25° C., and collecting precipitate that had formed therein.

The present invention significantly differs from the invention described in Patent Document 2 above. One reason is that, in the present invention, “collecting matter floating on or in the solution or adhering to the wall surface of a vessel” and treating the collected matter per se so as to obtain a glucan-protein complex is carried out instead of “removing matter floating on or in the solution or adhering to the wall surface of a vessel.” Another reason is that the obtained glucan-protein complex was found to have immunopotentiating activity and tumor growth inhibitory activity as a result of promotion of TNF-α production.

In the present invention, all types of Grifola including “Maitake” (Grifola frondosa), “Shiromaitake” (Grifola albicans Imaz.), “Choreimaitake” (Dendropolyporus umbellatus), and the like can be used. Fresh Grifola can be used as it is or after cutting it into pieces if necessary. Also, dried Grifola can be used as it is, after cutting it into pieces if necessary, or in a powdered form.

The method of thermal extraction with water is carried out at 50° C. to 135° C. for 15 minutes to 3 hours. For rapid extraction, this method is carried out under pressure at 100° C. or more, for example, at 1 to 2 atmospheric pressure at about 120° C. in a pressure pot for 30 minutes to 1 hour or thereabout.

As water, distilled water, purified water, ion exchanged water, tap water, and the like are used. About 4 to 20 parts by volume of water are used per part by weight of dried Grifola. When fresh Grifola is used, about 2 to 10 parts by volume of water are used per part by weight of Grifola.

As an alcohol used in steps (1): 2) and 4) above, methanol, ethanol, propyl alcohol, and the like can be used. Such alcohol is added to the extract to a final concentration between 20% and 70% by volume in step (1): 2). An alcohol with water content between 0% and 50% can be used. After the addition of the alcohol, the resulting solution is allowed to stand at a temperature between 1° C. and 25° C. for 1 hour to 20 hours, whereby matter floating on or in the solution or adhering to the wall surface of a vessel appears. The matter is then collected from the solution by filtration, pipetting, straining out with a meshed material, or similar methods.

The matter collected in step (1): 2) above is dissolved in water. A non-adsorbed fraction thereof is collected by anion-exchange column chromatography.

Alcohol is added to the solution obtained in step (1): 3) above to a final concentration between 20% and 50% by volume. The resulting solution is allowed to stand at a temperature between 1° C. and 25° C., whereby precipitate forms therein so as to be removed. Further, alcohol is added to the solution to a final concentration between 40% and 99% by volume. The resulting solution is allowed to stand at a temperature between 1° C. and 25° C., whereby precipitate forms therein so as to be collected. The precipitate collected is precipitate that has formed after the addition of high concentration alcohol.

The objective substance of the present invention obtained above is described as follows:

Appearance: an achromatic to brownish-colored liquid or solid;

Color reaction: positive in anthrone reaction and ninhydrin reaction;

Properties of aqueous solution: neutral to weakly acidic; and

Molecular weight: 10,000 to 150,000.

Based on the results of analysis of the substance obtained in the present invention, the substance mainly comprises glucan and protein. After the purification of the substance by column chromatography, it was confirmed that an antitumor substance having immunopotentiating activity obtained by the present invention mainly comprises a glucan-protein complex. The glucan to protein ratio thereof mainly ranges from 70:30 to 99:1. The ratio changes depending on product quality of Grifola as a starting material, conditions of extraction and purification, and the like.

As is apparent from the following examples, since the glucan-protein complex of the present invention is a substance having an immunopotentiating effect and an antitumor effect, it may be administered directly to humans or animals. Such substance may be added to beverages and foods, or feeds, whereby immunopotentiating activity or antitumor activity can be provided.

When the glucan-protein complex of the present invention is used as an immunopotentiating agent or an antitumor agent, carriers, excipients, and other additives generally used in pharmaceutical formulations may be added thereto. The immunopotentiating agent or antitumor agent of the present invention can be administered orally or parenterally. Particularly preferably, the agent is administered orally. Dosage forms of the agent for oral administration can be tablets, granules, powders, pills, capsules, solutions, syrups, and the like.

The dose of the immunopotentiating agent or antitumor agent of the present invention differs depending on each case in terms of age, weight, the route of administration, the administration frequency, and condition. Persons skilled in the art can adequately determine the dose in response to individual cases, although a typical dose thereof cannot easily be determined.

The immunopotentiating agent or antitumor agent of the present invention may be administered in a single dose. However, since the agent is a highly safe substance extracted from edible mushrooms, it can be administered repeatedly to humans or animals over a long period of time.

The dose of the glucan-protein complex of the present invention added to beverages and foods differs depending on each case in terms of age, weight, the intake frequency, and tumor condition. However, persons skilled in the art can adequately determine a specific dose thereof.

Beverages and foods in the present invention are not limited to beverages such as milk or other drinks, or to foods for daily consumption. They include all types of foods such as general beverages and foods including so-called healthy foods; foods with health claims including foods with nutrient function claims, foods for specified health use, and the like; and special-use foods including foods for patients, foods for the elderly, and the like.

According to the present invention, potentiating effects due to immunopotentiating activity or tumor growth inhibitory activity are provided using a glucan-protein complex having a molecular weight of 10,000 to 150,000, which is newly obtained by fractionation of hot water extract of Grifola.

### DESCRIPTION OF THE PREFERRED EMBODIMENTS

Hereafter, the present invention will be specifically described by referring to the following examples.

### Examples 1

Extraction Method

Fruit bodies of dried Maitake (Grifola frondosa) (500 g) were thermally extracted with 5 liters of distilled water at 120° C. for 60 minutes. Ethanol was added to 950 ml of the obtained soluble fraction to a final concentration of 50% by volume. The resulting solution was allowed to stand at 4° C. for 12 hours, whereby highly viscous brownish-red matter floating on or in the solution or adhering to the wall surface of a vessel was generated. After collecting the matter by pipetting or the like, the matter was dissolved in water. Thereafter, the non-adsorbed fraction thereof was collected by anion-exchange column chromatography using, for example, a Tris-HCl buffer solution (pH 8.2) as an eluate.

Alcohol was added to the solution to a final concentration between 20% and 50% by volume. The resulting solution was allowed to stand at a temperature between 1° C. and 25° C., whereby precipitate formed therein. After removing the precipitate, alcohol was added thereto to a final concentration between 40% and 99% by volume. The resulting solution was allowed to stand at a temperature between 1° C. and 25° C. so as to obtain precipitate that formed therein. When the thus obtained substance was subjected to anthrone reaction and ninhydrin reaction, positive results were obtained in each reaction. Then, the substance was purified by column chromatography and was found to be a glucan-protein complex. The glucan to protein ratio thereof was 91:9.

As a result of high-performance liquid chromatography analysis of the molecular weight distribution of the complex, the distribution of the molecular weight was found to be between 10,000 and 150,000.

As a result of a qualitative test of neutral monosaccharides by thin-layer chromatography, following acidolysis of glucan, only glucose was detected. In addition, after β-1,6 glucanase and β-1,3 glucanase were allowed to act on glucan, glucose was detected as a degradation product in each case.

### Example 2

Examination of Antitumor Activity

The substance obtained in Example 1 and normal saline solution as a control, respectively, were intraperitoneally administered 10 times to C3H mice, to which MM-46 carcinoma had been implanted, in an amount of 4 mg/kg of body weight. The activity on tumor growth of the substance obtained in Example 1 was examined. The results listed in table 1 were obtained.

TABLE 1

| | Tumor Growth |
|---|---|
| | Inhibitory Rate (%) |
| | 26 days after implantation |
| Control Group | 0.0 |
| (treated with normal saline solution) | |
| Treatment Group | 70.3 |
| (treated with the substance obtained in | |
| Example 1) | |

(n = 3 mice per group)

The tumor growth inhibitory rate was obtained by the following equation:
Tumor Growth Inhibitory Rate={1−(Average Tumor Weight of Treatment Group (g)/Average Tumor Weight of Control Group (g))}×100.

As is apparent from table 1, stronger tumor growth inhibitory effects were exhibited in the group to which the substance obtained in Example 1 had been administered compared with those exhibited in the control group. In addition, to examine the activation of immunocompetent cells, J774.1 cells, which were of a murine peripheral blood-derived macrophage-like cell line, were stimulated with the substance at 37° C. for 18 hours under the condition of 5% CO₂. The resulting amounts of TNF-α produced are listed in Table 2. In addition, the amount of TNF-α produced is a representative index indicating activation of immunocompetent cells.

TABLE 2

| | Amounts of TNF-α Produced (ng/ml) | | | | | | | |
|---|---|---|---|---|---|---|---|---|
| | 6 h | 12 h | 18 h | | | 24 h | | |
| Control Group | 19.0 ± 4.2 | 23.2 ± 0.8 | 33.0 ± 3.7 | | | 58.6 ± 2.6 | | |
| | | | | {close oversize brace} | * | | {close oversize brace} | * |
| Treatment Group | 14.0 ± 0.5 | 31.9 ± 3.0 | 43.8 ± 3.5 | | | 75.2 ± 7.0 | | |
| (treated with 250 μg/ml | | | | | | | | |
| of the substance | | | | | | | | |
| of Example 1) | | | | | | | | |
| Treatment Group | 13.8 ± 0.7 | 32.6 ± 4.7 | 53.2 ± 1.4 | | | 96.1 ± 10.6 | | |
| (treated with 500 μg/ml | | | | | | | | |
| of the substance | | | | | | | | |
| of Example 1) | | | | | | | | |

(n = 3 *Scheffe's test: significant difference confirmed within 5%.)

Based on the above results, it was confirmed that stronger tumor growth inhibitory effects and immunopotentiating activity were exhibited in the group to which the substance obtained in Example 1 had been administered compared with those exhibited in the control group.

What is claimed is:

1. A glucan-protein complex produced by the following (1) to (5):
(1) a step of thermally extracting mycelia or fruit bodies of Grifola with water;
(2) a step of adding alcohol to the obtained water-soluble extract fraction to a final concentration between 20% and 70% by volume, allowing the resulting solution to stand at a temperature between 1° C. and 25° C., and collecting matter floating on or in the solution or adhering to the wall surface of a vessel;
(3) a step of dissolving the collected matter in water and collecting non-adsorbed fraction by anion-exchange column chromatography;
(4) a step of adding alcohol to the resulting solution to a final concentration between 20% and 50% by volume, allowing the solution to stand at a temperature between 1° C. and 25° C., and removing precipitate that had formed therein; and
(5) a step of further adding alcohol to the resulting solution to a final concentration between 40% to 99% by volume, allowing the resultant to stand at a temperature between 1° C. to 25° C., and collecting precipitate that had formed therein.

2. The glucan-protein complex according to claim 1, the glucan to protein ratio of which is between 70:30 and 99:1.

3. The glucan-protein complex according to claim 1 or claim 2, which is obtained from “Maitake” (Grifola frondosa), “Shiromaitake” (Grifola albicans Imaz.), or “Choreimaitake” (Dendropolyporus umbellatus).

4. An immunopotentiating agent containing, as an active ingredient, the glucan-protein complex according to claim 1, claim 2, or claim 3.

5. An antitumor agent containing, as an active ingredient, the glucan-protein complex according to claim 1, claim 2, or claim 3.

6. Beverages and foods containing the glucan-protein complex according to claim 1, claim 2, or claim 3.
